(19) United States
(12) Patent Application Publication
(10) Pub. No.: US 2024/0157061 A1
(43) Pub. Date: May 16, 2024

(54) A safety needle device

(71) Applicant: HTL-STREFA S.A., Ozorków (PL)

(72) Inventors: Robert OSAK, Ozorków (PL); Marcin KOMUDA, Ozorków (PL); Marcin NIEMIEC, Ozorków (PL); Robert GRZELAK, Ozorków (PL); Rafal PERLAK, Ozorków (PL); Bartlomiej BIALAS, Ozorków (PL)

(21) Appl. No.: 18/284,340

(22) PCT Filed: Mar. 29, 2022
(86) PCT No.: PCT/EP2022/058333

§ 371 Date: Sep. 27, 2023

(30) Foreign Application Priority Data
Apr. 1, 2021 (EP) 21166669.8

(51) Int. Cl. A61M 5/32

(52) U.S. Cl. CPC A61M 5/326; A61M 5/3271

(57) ABSTRACT

Safety needle device (1) comprising: a hub (10) configured to be connected to a pen-injector, the hub (10) comprising a lateral wall (11) and an upper wall (12); a needle (20) attached to the hub (10), a back-end shield (30) slidable along the longitudinal direction (X-X) within the cavity (13) of the hub (10) and rotatable about the longitudinal direction (X-X), the back-end shield (30) being movable irreversibly from an initial position, before connection of the pen injector to the hub (10), to a locking position, after removal of the pen injector to the hub (10) passing through an intermediate position, an elastic member (40) arranged within the cavity (13) and acting between the upper wall (12) and the back-end shield (30) to urge the back-end shield (30) towards the proximal end (21) of the needle (10) along the longitudinal direction (X-X), a locking arrangement (50) comprising at least a first locking member (60) formed on the back-end shield (30) and at least a second locking member (70), the second locking member (70) cooperating with the first locking member (60) to guide the back-end shield (30) from the initial position to the locking position and to lock the back-end shield (30) in the locking position,

### FIELD OF INVENTION

The invention relates to the technical field of a medical device, in particular to a protection device able to prevent the user from accidental or intentional injuries with a needle before and after the use of the medical device.

### BACKGROUND OF THE INVENTION

Typically, a safety needle device comprises an elongated needle having a first distal end insertable into the patient's skin and a second proximal end insertable into a pen injector for supplying a substance. The needle is embedded in a hub and partially surrounded by a housing fixed to the hub. A known device comprises also a front-end shield and a back-end shield surrounding the needle and moving along the needle to protect the needle end before and after use. The shields are activated by means of spring elements interposed between the hub and the relative shield. The activation of the front-end shield occurs with the injection, while the activation of the back end shield occurs when coupling of the safety device with a pen injector. Upon activation, the shields pass into a protect configuration that prevents further uses or injuries. In detail, the device comprises locking means able to lock the sliding of the front and back-end shields after use.

For example, in the art elastic clamps engage the back-end shield when it reaches a specific position sliding towards the proximal end of the needle. Other known locking means are flexible tabs formed on the hub that engage the back-end shield to prevent further movements of it after use.

However, the known safety needle devices show some drawbacks. In fact, such devices do not ensure the correct protection to the user from further uses and from accidental injuries. Furthermore, known devices are complex since they require a lot of elements in order to achieve the correct protection of the user, thereby increasing the overall costs of production.

Furthermore, the structural complexity of known safety needle devices increases the production costs and the assemblage time.

### SUMMARY OF THE INVENTION

In this context, the technical task underlying the present invention is to propose a safety needle device which overcomes the drawbacks of the above prior art.

Specifically, it is an object of the present invention to provide a safety needle device able to ensure the protection to the user and at the same time to improve the availability of the device itself and to reduce the complexity of the back-end mechanism.

The technical task set out and the specified objects are substantially achieved by a cooperation of protrusions formed on the back-end shield and passing through guides formed on the hub to guide and lock the back-end shield.

### BRIEF DESCRIPTION OF THE DRAWINGS

The present invention will now be described in more detail hereinafter with reference to the accompanying drawings, in which some embodiments of the invention are shown.

FIG. 1 is a section view of the safety needle is a lateral view of the safety needle device according to a first embodiment of the present invention, in pre-use configuration;

FIG. 2 is a different section view of the safety needle of FIG. 1;

FIG. 3 is a section view of the safety needle device according to the embodiment of FIG. 1, in a use configuration;

FIG. 4 is a different section view of the safety needle device of FIG. 3;

FIG. 5 is a section view of the safety needle device according to the embodiment of FIG. 1, in a locking configuration;

FIG. 6 is a section view of the safety needle device according to the embodiment of FIG. 1, in an after-use configuration;

FIG. 7 is a different section view of the safety needle device of FIG. 6;

FIG. 8 is a lateral view of the safety needle device according to the embodiment of FIG. 1 where some elements are hided to better show other aspects;

FIG. 9 is a perspective view of an element of safety needle device of FIG. 1;

FIG. 10 is a section view of the safety needle device according to a second embodiment of the present invention, in pre-use configuration;

FIG. 11 is a different section view of the safety needle of FIG. 10;

FIG. 12 is a section view of the safety needle device according to the embodiment of FIG. 10, in a use configuration;

FIG. 13 is a different section view of the safety needle device of FIG. 10;

FIG. 14 is a section view of the safety needle device according to the embodiment of FIG. 10, in a locking configuration;

FIG. 15 is a section view of the safety needle device according to the embodiment of FIG. 10, in an after-use configuration;

FIG. 16 is a different section view of the safety needle device of FIG. 15;

FIG. 17 is a lateral view of the safety needle device according to the embodiment of FIG. 10 where some elements are hided to better show other aspects;

FIG. 18 is a perspective view of an element of safety needle device of FIG. 10;

FIG. 19 is a perspective view of an element of safety needle device of FIG. 1 and FIG. 10.

### DETAILED DESCRIPTION

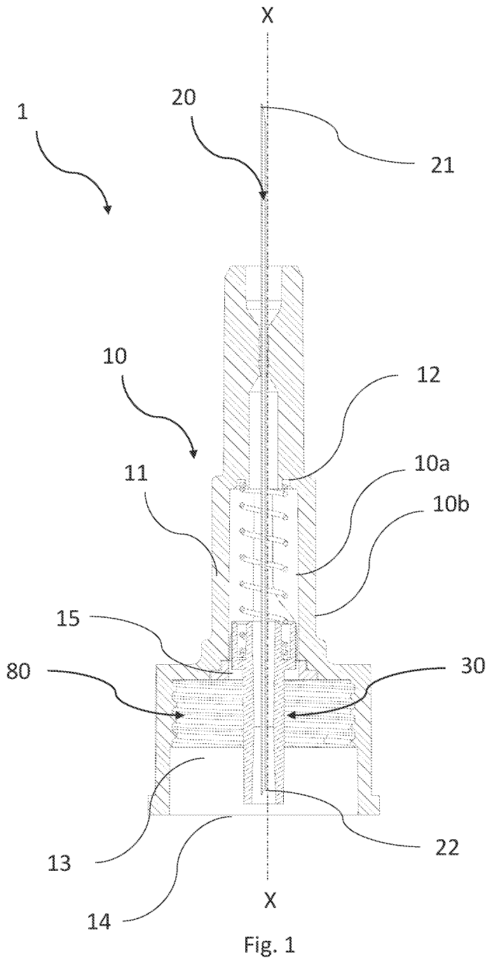

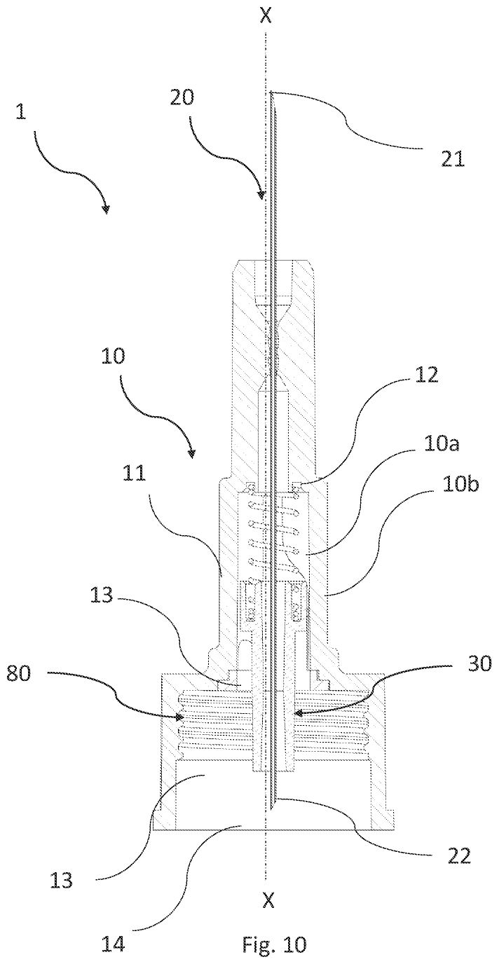

With reference to FIGS. 1-8 and 10-17, a safety needle device is indicated as a whole by numeral 1. Specifically, the FIGS. 1-8 and 10-17 do not show a front-end mechanism but they are focused on a back-end mechanism. Safety needle device 1 can be associated to a pen injector, not shown, containing a substance to inject into a patient. It is to be noted, as clarify in the following, that FIGS. 1-9 show a first embodiment of the safety needle device 1 and FIG. 10-18 show a second embodiment of the safety needle device 1.

The safety needle device 1 comprises a hub 10 configured to be connected to the pen injector. The hub 10 comprises a lateral wall 11 and an upper wall 12 which define therebetween a cavity 13. The latest extends between the upper wall 12 and a lower aperture 14 configured to receive the pen injector.

The hub 10 has an intermediate aperture 15 defined between the upper wall 12 and the lower aperture 14.

Preferably the hub 10 has an inner surface 10a facing to the cavity 13 and opposed outer surface 10b.

More preferably, the hub 10 comprises retaining means 80 configured to retain the pen injector. The retaining means 80 are formed on the inner surface 10a of the lateral wall 11 near the aperture 14.

According to a preferred embodiment, the hub 10 has a tubular shape with a circular cross section. Preferably, the intermediate aperture 15 has a radius shorter than the radius of the lower aperture 14.

The safety needle device 1 comprises a needle 20 attached to the hub 10. The needle 20 extends along a longitudinal direction X-X between a distal end 21, formed to be inserted into a patient, and opposed proximal end 22 formed to be connected to the pen injector.

According to one embodiment, at least a portion of the needle 20 is arranged inside the cavity 13. Preferably, the proximal end 22 of the needle 20 is arranged inside the cavity 13.

The safety needle device 1 comprises a back-end shield 30 slidable along the longitudinal direction X-X within the cavity 30 of the hub 10 and rotatable about the longitudinal direction X-X. Specifically, the back-end shield 30 is slidable and rotatable with respect to the hub 10. Preferably, the back-end shield 30 is movable irreversibly from an initial position, before connection of the pen injector to the hub 10, to a locking position, after removal of the pen injector to the hub 10 passing through an intermediate position. More preferably, during the connection of the hub 10 to the pen injector, the back-end shield 30 passes from the initial position to the intermediate position sliding and rotating towards the distal end 22 of the needle 20 along and about the longitudinal direction X-X. Moreover, during the removal of the pen injector the back-end shield 30 passes from the intermediate position to the locking position sliding and rotating towards the proximal end 21 of the needle 10 along the longitudinal axis X-X.

According to a preferred embodiment, the back-end shield 30 is configured to cover and protect the proximal end 22 of the needle 30 in the locking position. Specifically, the back-end shield 30 is configured to be locked in the locking position avoiding further sliding that would lead to uncovering the proximal end 22 of the needle 20 after use.

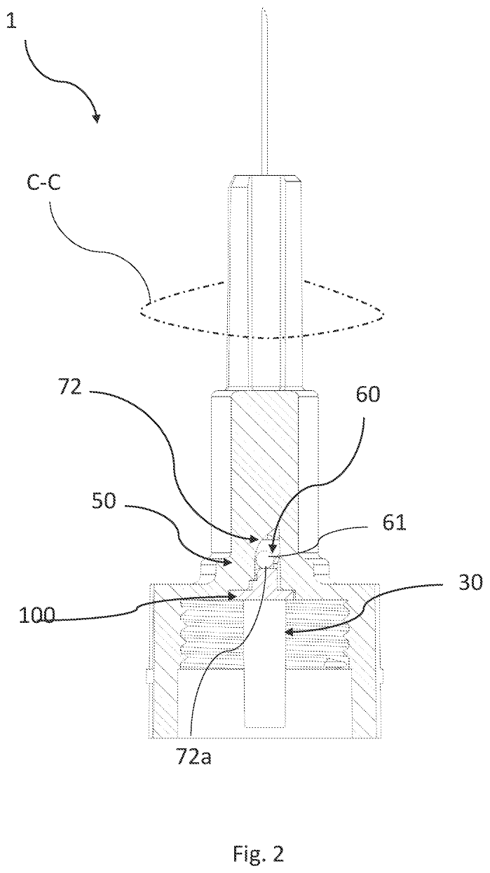

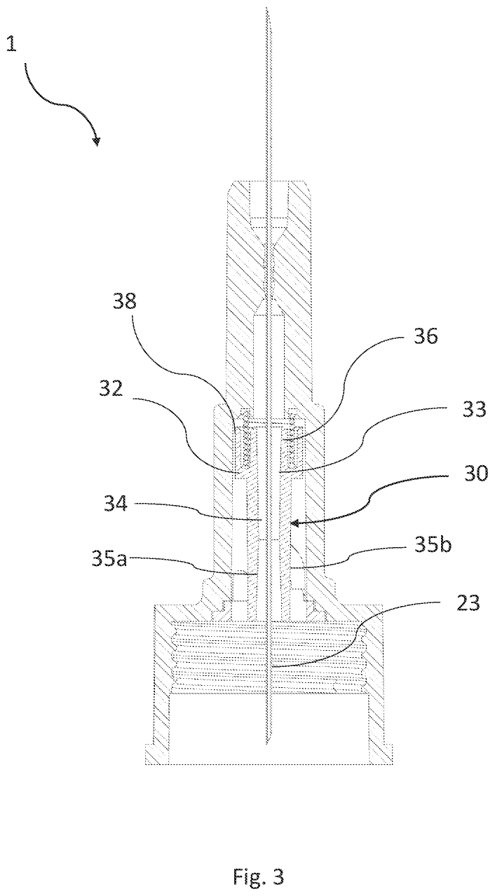

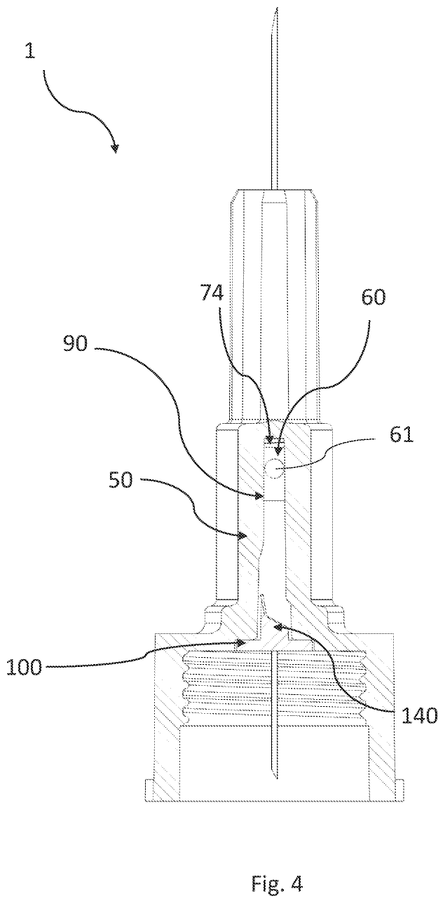

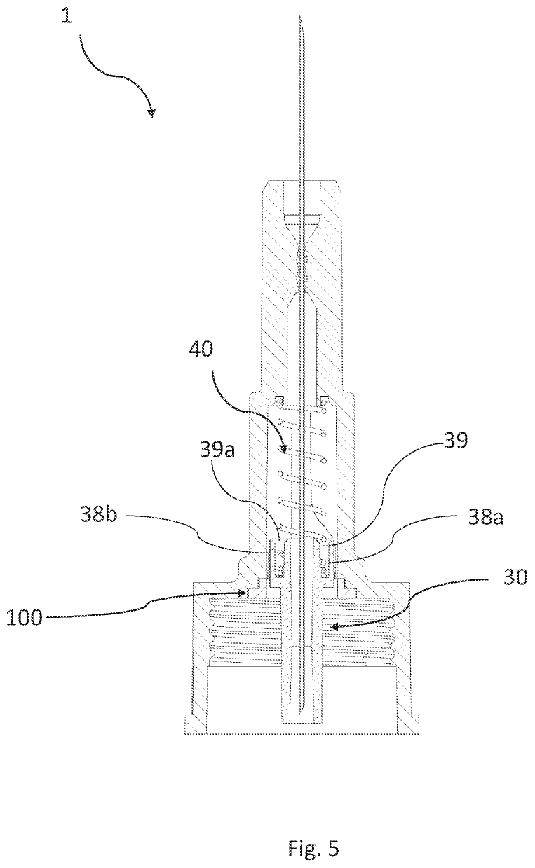

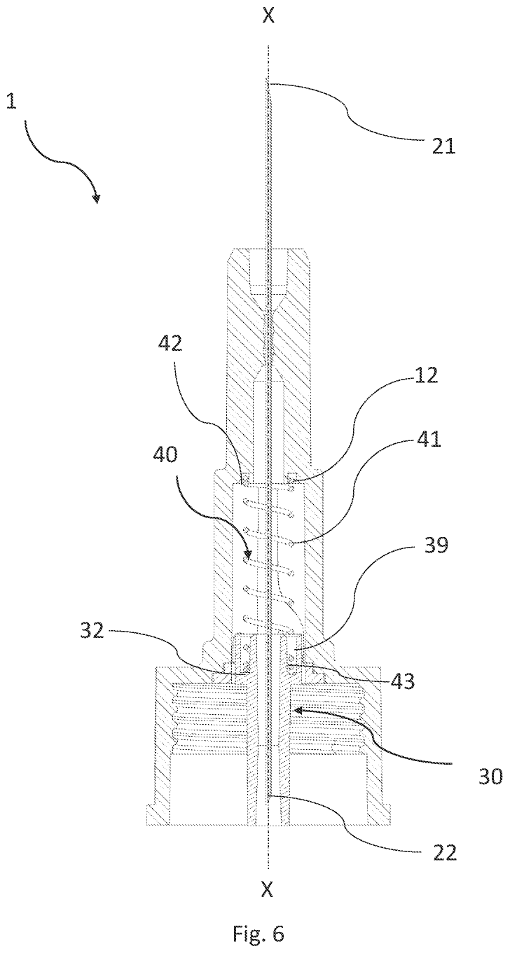

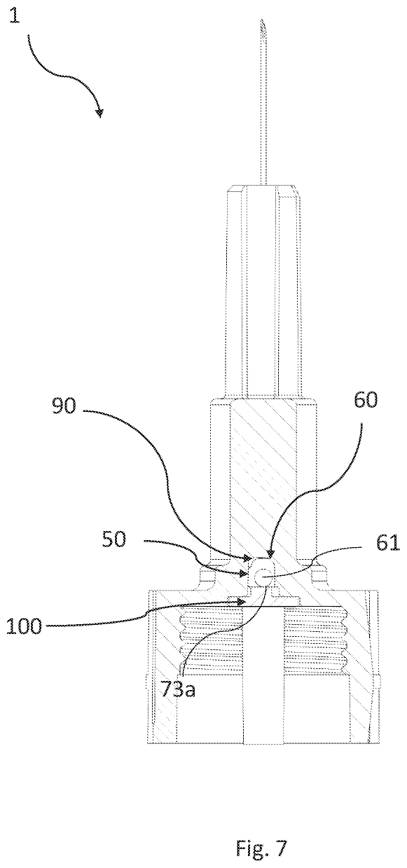

According to the first embodiment, the back-end shield 30, as shown in FIGS. 1 and 2, in the initial position covers the proximal end 22 of the needle 20. Subsequently, the back-end shield 30, as shown in FIGS. 3 and 4, in the intermediate position leaves uncovered a first portion 23 of the needle 20 arranged inside the cavity 13 and also the proximal end 22 of the needle 20. Finally, the back-end shield 30, as shown in FIG. 5, in the locking position covers the proximal end 22 of the needle 20. It is worth noting that upon reaching the locking position the back-end shield 30 is configured to cover the proximal end 22 of the needle 20. Optionally, in the locking position a limited sliding the back-end shield 30 along the longitudinal direction X-X is also allowed without uncovering the proximal end 22 of the needle 20 in FIGS. 6 and 7 for example after removing the pen injector.

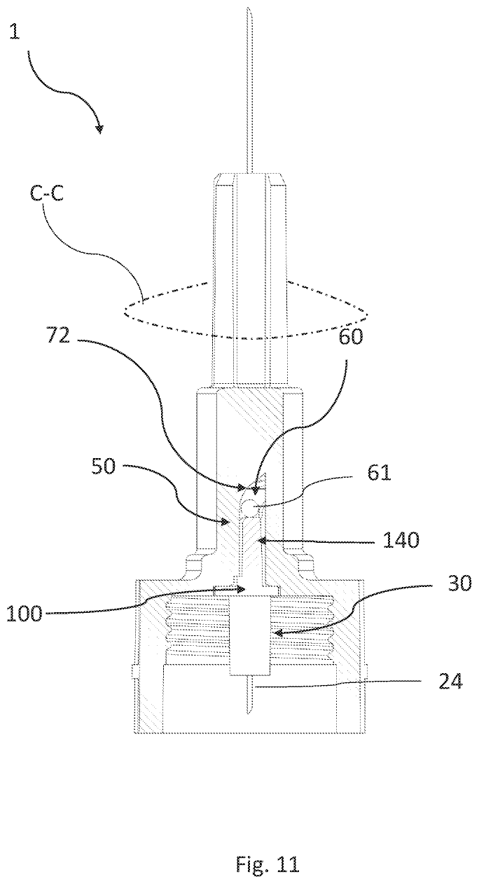

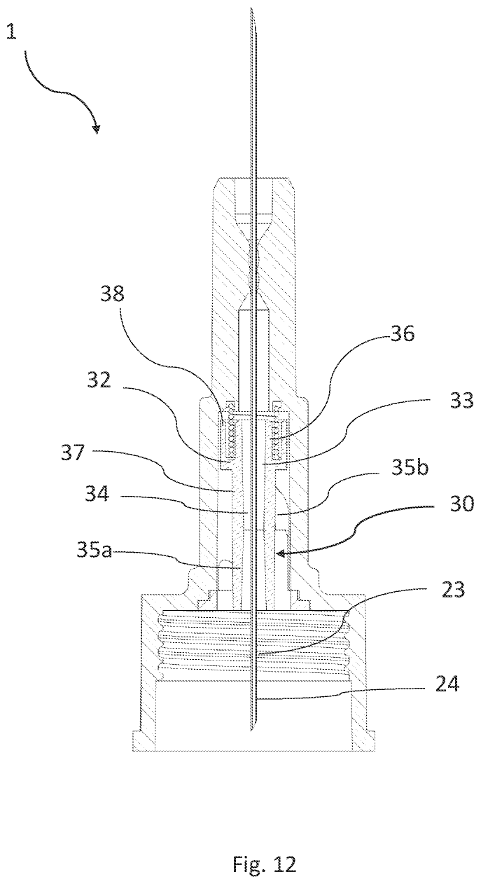

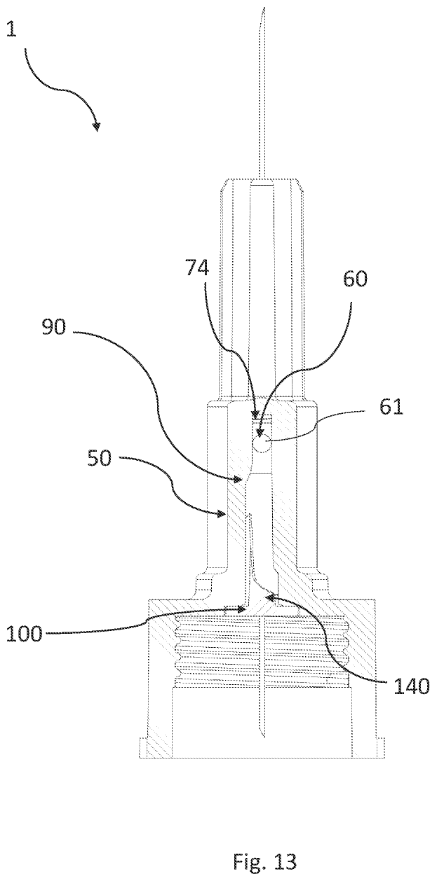

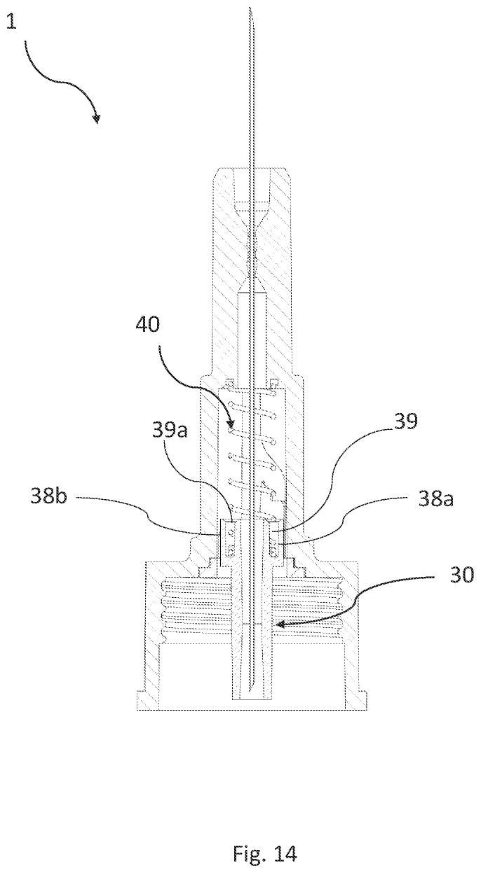

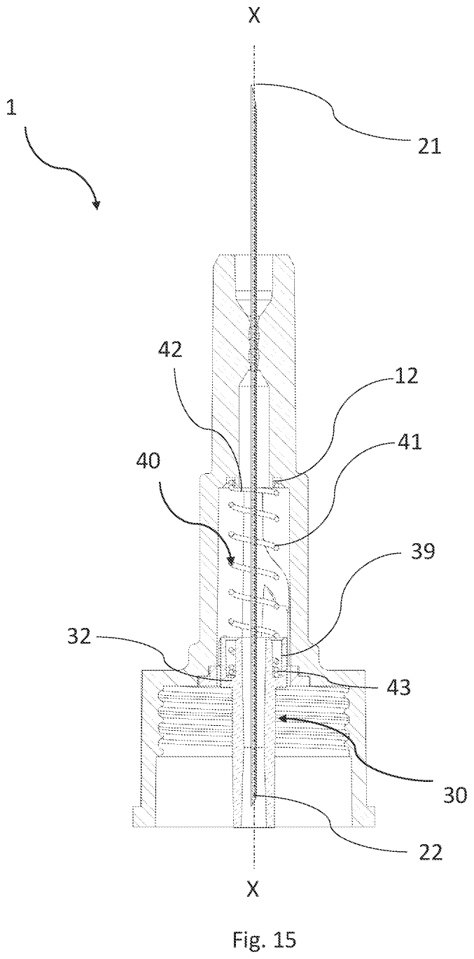

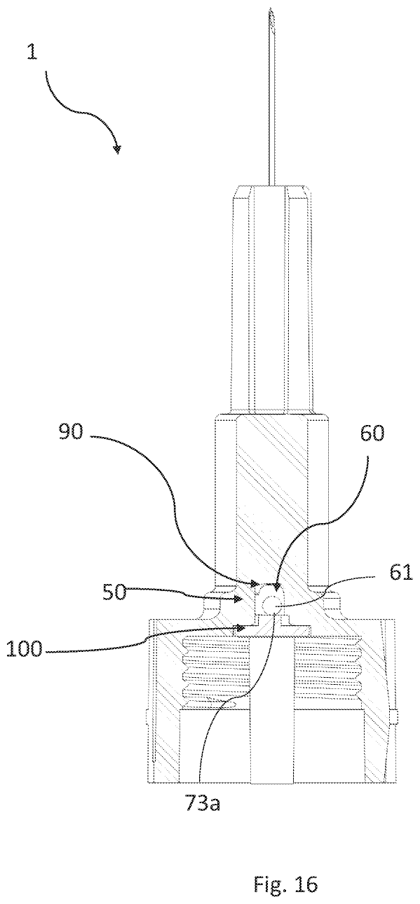

According to the second embodiment, the back-end shield 30, as shown in FIGS. 10 and 11, in the initial position leaves uncovered a second portion 24 of needle 20 arranged inside the cavity 13 and the proximal end 22 of the needle 20. Subsequently, the back-end shield 30, as shown in FIGS. 12 and 13, in the intermediate position leaves uncovered a first portion 23 of the needle 20 arranged inside the cavity 13 and also the proximal end 22 of the needle 20. It is to be noted that the second portion 24 being shorter than the first portion 23 because the back-end shield 30 has been moved towards the distal end 21 of the needle. Finally, the back-end shield 30, as shown in FIG. 14, in the locking position covers the proximal end 22 of the needle 20. It is worth noting that upon reaching the locking position the back-end shield 30 is configured to cover the proximal end 22 of the needle 20. Optionally, in the locking position a limited sliding the back-end shield 30 along the longitudinal direction X-X is also allowed without uncovering the proximal end 22 of the needle 20 in FIGS. 15 and 16, for example after removing the pen injector.

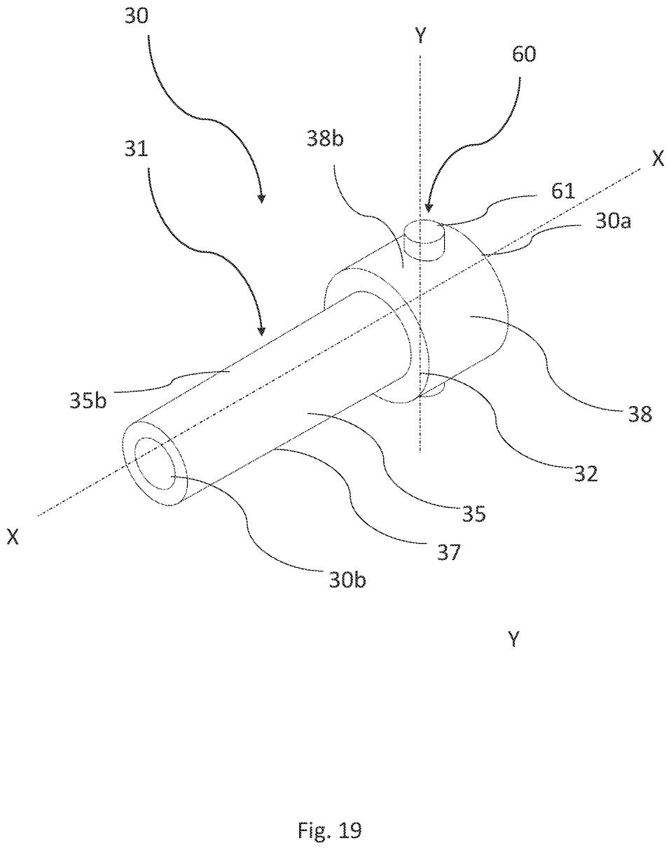

According to one embodiment, the back-end shield 30 (FIG. 19) comprises an hallow body 31 extending between a distal portion 30a facing to the upper wall 12 of the hub 10 and a proximal portion 30b facing to the lower aperture 14 of the hub 10. Specifically, the back-end shield 30 is arranged inside the cavity 13. The hallow body 31 comprises a lower wall 32 formed proximal to the distal portion 30a. Such lower wall 32 has an opening 33 which is configured to allow a portion of the needle 20 to pass through while the back-end shield 30 is moving between the initial position and locking position. The hallow body 31 further comprises a lateral wall 35 extending from the lower wall 32 along the longitudinal direction X-X and defining a passing through channel 34. The passing through channel 34 is configured to house at least partially the needle 20 arranged inside the cavity 13. The lateral wall 35 has an inner surface 35a facing to the passing through channel 34 and an outer surface 35b facing to the cavity 13 of the hub 10.

According to one embodiment, the lateral wall 35 has a distal portion 36 extending along the longitudinal direction X-X from the lower wall 32 towards the distal end of the needle 20 and a proximal portion 37 extending along the longitudinal direction X-X from the lower wall 32 towards the proximal end of the needle 20. Preferably, the hallow body 31 comprises a retaining wall 38 extending along the longitudinal direction X-X from the lower wall 32 towards the distal end of the needle 20 and surrounding at least partially the lateral wall 35 preferably the distal portion 36 of lateral wall 35. Specifically, the lower wall 32, the retaining wall 38 and the distal portion 36 of the lateral wall 35 define the annular chamber 39 extending from the lower wall 32 to an annular aperture 39a. It is to be noted that, the retaining wall 38 has an inner surface 38a facing to the surrounded portion of the lateral wall 35, preferably facing to the distal portion 36 of the lateral wall 35, and an outer surface 38b facing to the cavity 13, preferably the inner surface of the hub 10.

The safety needle device 1 comprises an elastic member 40 arranged within the cavity 13 and acting between the upper wall 12 and the back-end shield 30 to urge the back-end shield 30 towards the proximal end 21 of the needle 10 along the longitudinal direction X-X. Preferably, the elastic member 40 is configured to constantly urge the back-end shield 30 towards the proximal end 22 of the needle 10. Specifically, the elastic member 40 generates a force directed to the proximal end 22 of the needle 20 as a result of a longitudinal motion of the back-end shield 30 towards the distal end 22 of the needle 20. In detail, the elastic member 40 is configured to be elastically compressed during the coupling of the pen injector with the hub 10, namely during the passage of the back-end shield 30 from the initial position to the intermediate position and to be elastically extended during the removal of the pen injector from the hub 10 with the passage of the back-end shield 30 from the intermediate position to the locking position. In other words, the pen injector acts on the hallow body 31 at the proximal portion 30b causing the motion of the back-end shield 30 from the initial position to the intermediate position and then the compression of the elastic member 40. After the removal of the pen injector and its action on the lower wall 32, the back-end shield 30 passes from the intermediate position to the locking position by means of elastic member 40 extension.

Preferably, the elastic member 40 is interposed between the hub 10 and the back-end shield 30 in order to constantly urge the back-end shield 30. Specifically, the elastic member 40 is at least partially arranged inside the annular chamber 39 which is configured to accommodate a portion of the elastic member 40. In details, the elastic member 40 is interposed between the upper wall 12 and the lower wall 32 in order to constantly urge the back-end shield 30 towards the proximal end 22.

It is to be noted that, the elastic member 40 surrounds at least partially the needle 20 passing inside the cavity 13 of the hub 10.

According to a preferred embodiment, the elastic member 40 comprises a spring member 41 which extends between a distal portion 42 and a proximal portion 43 along the longitudinal axis X-X. Accordingly, the portion 42 abuts against the upper wall 12 and the proximal portion 43 abuts against the lower wall 32.

The safety needle device 1 comprises a locking arrangement 50 configured to guide the back-end shied 30 from the initial position to the locking position and to lock the back-end shield 30 in the locking position. Preferably, the locking arrangement 50 is configured to rotate the back-end shield 30 upon sliding of the back-end shield 30 along the longitudinal direction X-X and to lock the back-end shield 30 in order to constantly cover the proximal end 22 of the needle 20.

The locking arrangement 50 comprises at least a first locking member 60 formed on the back-end shield 30 and at least a second locking member 70. Specifically, the second locking member 70 cooperates with the first locking member 60 to guide the back-end shield 30 from the initial position to the locking position and to lock the back-end shield 30 in the locking position.

The second locking member 70 comprises a first guide element 90 formed on the lateral wall 11 of the hub and a second guide element 100 attached to the lateral wall 11 inside the cavity 13 at the intermediate aperture 15. Specifically, the first guide element 90 and the second guide element 100 define a guide track 71 inside the cavity 13. The first locking member 60 comprises a protrusion 61 configured to engage the first guide element 90 and the second guide element 100 to slide along the guide track 71.

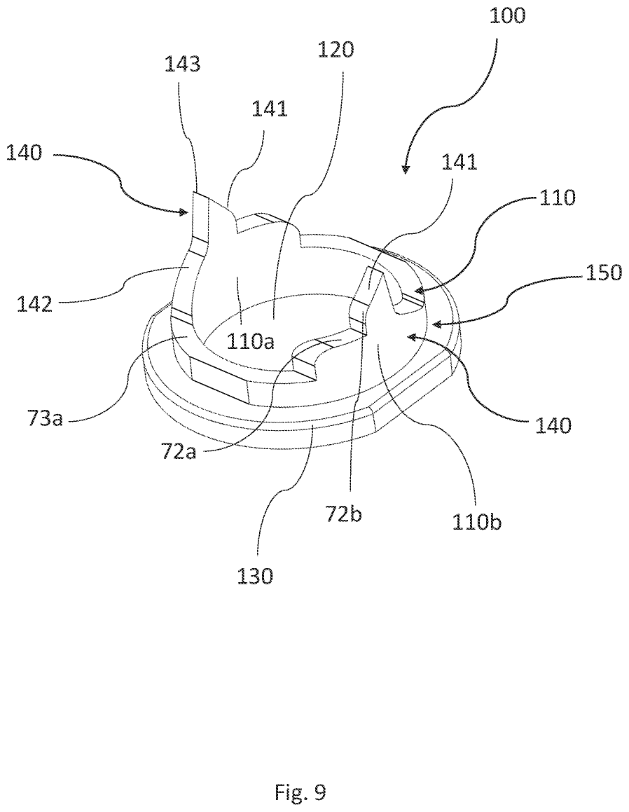

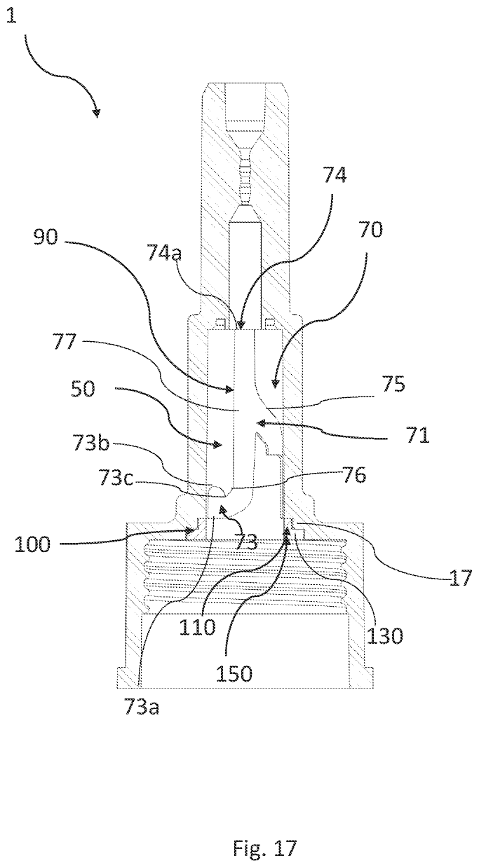

It is to be noted that the guide track 7171 has a shape shown in FIGS. 9 and 17.

According to a preferred embodiment, the protrusion 61 is formed on the outer surface 38b of the retaining wall 38 of the back-end shield 30, preferably projecting from the outer surface 38b along a transversal direction Y-Y perpendicular to the outer surface 38b and then to the longitudinal direction X-X. Preferably, the protrusion 61 is formed proximal to the distal portion 30a of the back-end shield 30. More preferably, the locking arrangement 50 comprises at least two protrusions 61 mutually spaced apart along the outer surface 38b of the retaining wall 38. Even more preferably, the two protrusions 61 are circumferentially spaced apart on the back-shield 30. The preferred embodiment shown in the figures, the protrusions 61 are symmetric and circumferentially spaced apart on the outer surface 35b of the back-end shield.

According to a preferred embodiment, the protrusions 61 are pins.

According to one embodiment, the first guide element 90 is formed on the lateral wall 11 of the hub 10. Preferably, the first guide element 90 comprises a groove formed on the inner surface 10a of the hub 10. More preferably, the hub 10 comprises two symmetric first guide elements 90 circumferentially spaced apart on the lateral wall 11. In other words, the hub 10 has two first guide elements 90 angularly spaced apart along the lateral wall 11.

Such configuration cooperating with the second guiding element 100 allows to correctly guide the back-end shield 30 between the initial position to the locking position and cooperates with the protrusions to lock the back-end shield 30 in the locking position.

According to one embodiment, the second guide element 100 comprises an annular portion 110 configured to engage the lateral wall 11 at the intermediate aperture 15. Specifically, the annular portion 110 defines a passing through channel 120 to allow the sliding of the back-end shield 30 along the longitudinal direction X-X. Preferably, the annular portion 110 comprises an inner surface 100a facing the back-end shield 30 and an outer surface 100b facing the lateral wall 11 of the hub 10. The annular portion 100 has flange 130 protruding from the outer surface 100b and configured to engage the lateral wall 11 of the hub.

According to one embodiment, the annular portion 110 has retaining means 150 configured to engage with the lateral wall 12 at the intermediate aperture 15. Specifically, the hub 10 comprises at intermediate aperture 15 a retaining groove 17 formed on the lateral wall 12. The retaining groove 17 has a circumferential profile and facing the lower aperture 13. The retaining means 150 are configured to engage the retaining groove 17 to attach the second guiding element 100 to the hub 10. Preferably, the retaining means 150 allows to engage the hub 10 by interference fit. More preferably, the annular portion 110 and the flange 130 are configured to be inserted into the retaining groove 17 and be attached to the hub by interference fit. It is to be noted that the retaining means 150 allow to remove the annular portion 110 from the hub 10.

The second guide element 100 further comprises at least a guiding wing 140, preferably two, projecting from the annular portion 110 towards the distal end 21 inside the cavity 13 and configured to engage the protrusion 61 and guide it from initial position to the locking position. Preferably, guiding wings 140 are angularly spaced apart along the annular portion 110. In other words, the guiding wings 140 are circumferentially spaced apart on the annular portion 110. It is to be noted that the guiding wing 140 follow a circumferential profile defined by the annular portion 110. In this way, the guiding wing 140 extends the passing through channel 120 in order to allow the sliding of the back-end shield 30 along the longitudinal direction X-X.

It is to be noted that upon attachment of the annular portion 110 to the hub the guiding wing 140 are inserted into the cavity 13 facing the upper wall 12.

According to one embodiment, the first guiding element 90 and the second guiding element 100 defines a first stopping area 72 and a second stopping area 73.

The first stopping area 72 is configured to engage the protrusion 61 in the initial position of the back-end shield 30 to prevent longitudinal sliding of the back-end shield 30 towards the proximal end 22 of the needle 20. Preferably, the first stopping area 72 has a first support portion 72a that retains the protrusion 61 and maintains the back-end shield 30 in the initial position and a second support portion 72b that prevents undesired rotation of the back-end shield 30 about the longitudinal direction X-X. More preferably the second support portion 72b extends along the longitudinal direction X-X from the first support portion 72a towards the distal end 22 of the needle 20. Even more preferably, the first support portion 72a and the second support portion 72b define an initial bay which retains the back-end shield 30 in the initial position until the coupling of the pen injector with the hub 10. As a matter of fact, the first stopping area 72 retains the back-end shield 30 against preloaded elastic force of the elastic member 40 which urges the back-end shield 30 towards the proximal end 22.

According to a preferred embodiment shown in the figures, the annular portion 110 defines the first support portion 72a and the guiding wing 140 defines at least partially the second support portion 72b.

The second stopping area 73 is configured to engage the protrusion 61 in the locking position of the back-end shield 30 to prevent longitudinal sliding of the back-end shield 30 both towards the distal end 21 and the proximal end 22 of the needle 10. Specifically, the second stopping area 73 is configured to retain the protrusion 61 in order to prevent further use of the safety needle device 1 and the sliding of the back-end shield along the longitudinal direction X-X. Preferably, the second stopping area 73 has a first support portion 73a and a second support portion 73b which prevent the longitudinal sliding of the back-end shield 30 both towards the distal end 21 and the proximal end 22 of the needle. In detail, also the first support portion 73a limits the stroke of the back-end shield 30 along the longitudinal axis X-X retaining the shield 30 against preloaded elastic force of the elastic member 40 which continues to urge the back-end shield 30 towards the proximal end 21 also in the locking position. More preferably, the second stopping area 73 has a third support portion 73c which prevent rotation of the back-end shield 30 about the longitudinal direction X-X. Specifically, the third support portion 73c extends from the second support potion 73b along the longitudinal direction X-X towards the proximal end 21 of the needle 20. Even more preferably, the support portion 73a, 73b, 73c of the second stopping member 72 defines a locking bay which engages and retains the protrusion 61 when the back-end shield 30 reaches the locking position. In this way the back-end shield is retained in the locking position and the proximal end 22 of the needle is covered after use of the safety needle device.

According to a preferred shown in the figures, the annular portion 110 comprises the first support portion 73a and the first guiding element 90 comprises the second support portion 73b and third support portion 73c.

In other words, the first stopping area 72 and the second stopping area 73 have substantially a “C” shape wherein the “C” of the first stopping is rotated ninety degrees counterclockwise.

It is to be noted that, the first stopping area 72 and second stopping area 73 are circumferentially spaced apart. More preferably, the first stopping area 72 and second stopping area 73 are spaced apart along a circumferential direction C-C defined around the hub 10

According to the preferred embodiment shown in FIGS. 1-9, the first stopping area 72 and the second stopping area 73 are arranged proximal to the intermediate aperture 15. Specifically, the first stopping area 72 and the second stopping area 73 are substantially on a same plane perpendicular to the longitudinal direction X-X. Accordingly, as reported above, in the initial position the back-end shield 30 covers the proximal end 22 of the needle 20. In detail, first support portion 72a of the first stopping area 72 and the first support portion 73a of the second stopping area 73 are on the same plane.

According to the preferred embodiment shown in FIGS. 10-18, alternative to the previous embodiment, the first stopping area 72 is arranged towards the distal end 21 of the needle 20 and the second stopping area 73 is arranged towards the proximal end 22 of the needle 20. Specifically, the first stopping area 72 and the second stopping area 73 are offset along the longitudinal direction X-X. In other words, the first stopping area 72 and the second stooping area 73 are mutually spaced apart along the longitudinal direction X-X, with the first stopping area 72 towards the distal end of the needle and the second stooping area 73 towards the proximal end of the needle.

In other words, first support portion 72a of the first stopping area 72 72 is arranged towards the distal end 21 of the needle 20, while the first support portion 73a of the second stopping area 73 arranged towards the proximal end 22 of the needle 20.

According to a preferred embodiment, the first guiding element 90 comprises a third stopping area 74 configured to engage the protrusion 61 in the intermediate position to prevent longitudinal sliding of the back-end shield 30 towards the distal end 22 of the needle 20. Preferably, the third stopping area 74 limits the stroke of the protrusion along the longitudinal direction X-X. More preferably, the first guiding element 90 comprises at the third stopping area 74 a support portion 74a configured to engage the protrusion 61 in the intermediate position to prevent longitudinal sliding of the back-end shield 30 towards the distal end 22 of the needle 20. Specifically, the protrusion 61 abuts against the support portion 74a of the third stopping member 74 in the intermediate position.

It is to be noted that the third stopping area 74 is spaced apart from the first stopping area 72 and from the second stopping area 73 along the longitudinal direction X-X towards the distal end 21 of the needle 20. At the same time, the third stopping area 74 is circumferentially spaced apart both from the first stopping area 72 and the second stopping member 73. Preferably, the third stopping area 74 is arranged in the middle between the first stopping area 72 and the second stopping area 73 along the circumferential direction C-C which goes around the hub 10.

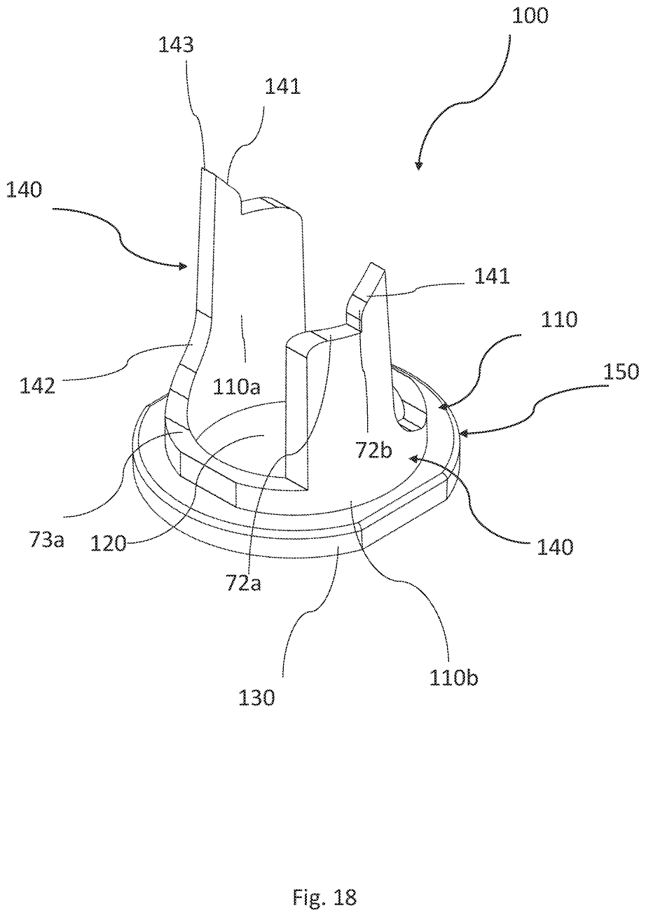

According to one embodiment, the guiding wing 140 comprises a first wing rotating portion 141 and a second wing rotating portion 142 configured to rotate the back-end shield 30 upon sliding along the longitudinal direction X-X cooperating with the first guiding element 90. Specifically, the first wing rotating portion 141 and a second wing rotating portion 142 (FIGS. 9 and 18) extends from the annular portion 110 along inclined direction with respect to the longitudinal direction X-X up to a connecting apex 143. In other words, the guiding wing 140 is tapered from the annular portion 110 towards the distal end along longitudinal direction X-X up to a connecting apex 143 defining on the opposed slopes the first wing rotating portion 141 and the second wing rotating portion 142.

The first wing rotating portion 141 is configured to guide and to rotate the back-end shield 30 upon sliding of the back-end shield 30 along the longitudinal axis X-X from the initial position towards the intermediate position. Preferably, the first wing rotating portion 141 is configured to engage the protrusion 61 and cooperating with the first guiding element 90 to rotate the back-end shield 30 upon sliding of back-end shield 30 along the longitudinal axis X-X from the initial position towards the intermediate position.

Instead, the second wing rotating portion 142 is configured to guide and rotate the back-end shield 30 upon sliding of the back-end shield 30 along the longitudinal axis X-X from the intermediate position towards the locking position. Preferably, the second wing rotating portion 142 is configured to engage the protrusion 61 and cooperating with the first guiding element 90 to rotate the back-end shield 30 upon sliding of back-end shield 30 along the longitudinal axis X-X from the intermediate position towards the locking position. More preferably, the second wing rotating portion 142 is arcuate and allows the rotation of the back-end shield 30 during the sliding along the longitudinal direction X-X. The combination of the rotation and the sliding allows the protrusion to engage the second stopping area 73.

According to a preferred embodiment, the first guiding element 90 comprises a first rotation portion 75 and a second rotation portion 76 configured to rotate the back-end shield 30 upon sliding along the longitudinal direction X-X cooperating with the second guiding element 100. Preferably, the first rotation portion 75 cooperates with the first wing rotating portion 141 and the second rotation portion 76 cooperates with the second wing rotating portion 142.

Specifically, the first rotation portion 75 is configured to engage the protrusion 61 and cooperating with the second guiding element 100, preferably with the first wing rotating portion 141, to rotate the back-end shield 30 upon sliding of back-end shield 30 along the longitudinal axis X-X from the initial position towards the intermediate position. Preferably the first rotation portion 75 is arcuate and allows the rotation of the back-end shield 30 during the sliding along the longitudinal direction X-X. The combination of the rotation and the sliding allows the protrusion 61 to get out from the first stopping area 72.

The second rotation portion 76 is configured to engage the protrusion 61 and cooperating with the second guiding element 100, preferably with the second wing rotating portion 142, to rotate the back-end shield 30 upon sliding of the back-end shield 30 along the longitudinal axis X-X from the intermediate position towards the locking position. Preferably, the second rotation portion 76 drives the protrusion 61 to the second wing rotating portion 142 allowing the rotation of the back-end shield 30 during the sliding along the longitudinal direction X-X. The combination of the rotation and the sliding allows the protrusion 61 to engage the second stopping area 73.

According to a preferred embodiment, the first guiding element 90 comprises a linear guiding portion 77 configured to engage the protrusion 61 and guide the back-end shield 30 along longitudinal axis X-X preventing rotation of the back-end shield 30 from the initial position towards the intermediate position and from the intermediate position towards the locking position. Preferably, the linear guiding portion 77 connects the first stopping area 72 and the second stopping area 73 with the third stopping area 74 such that to guide the protrusion 61 during the passage of the back-end shield 30 from the initial position to intermediate position and then from the intermediate position to the final position.

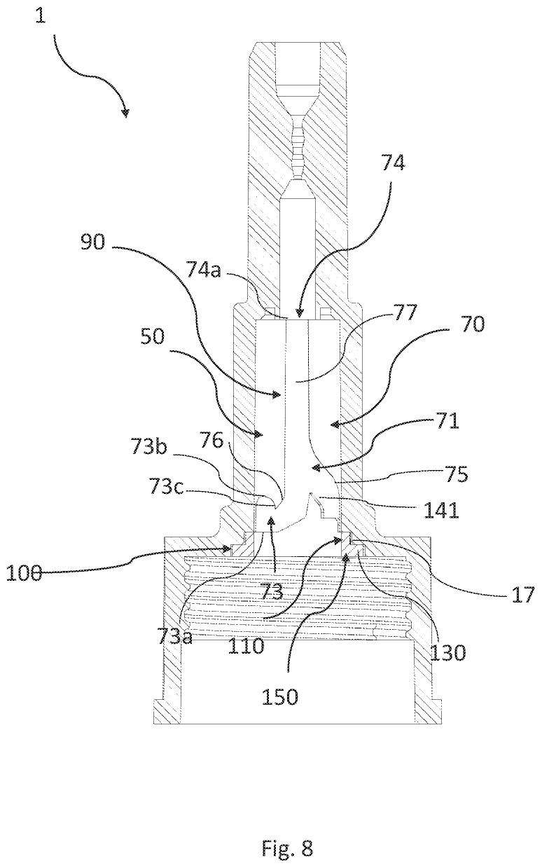

According to the preferred embodiments as shown in FIGS. 8 and 17, the first guiding element 90 and the second guiding element 100 define a guide track 71. Specifically, the guide track 71 extends from the first stopping area 72 defined by the combination of the annular portion 110 and a portion of the first guiding element 90, preferably the first rotation portion 75. Then, the guide track 71 has the linear guiding portion 77 which is connected to the first stopping area 72 by the first rotation portion 75 and the first wing rotating portion 141. In detail, the first rotation portion 75 and the first wing rotating portion 141 route the protrusion 61, rotating the back-end shield 30, from the first stopping area 72 to the linear guiding portion 77. The latter extends up to the third stopping area 73 wherein the protrusion 61 abuts in the intermediate position. The guide track 71 drives the protrusion 61 from the intermediate position to the locking position along the linear guiding portion 77 towards the proximal end of the needle. Preferably, the protrusion 61 slides along the linear guiding portion 77 two times in opposed verses along the longitudinal direction X-X:
towards the distal end of the needle during the sliding from the initial position to the intermediate position;
towards the proximal end of needle during the sliding from the intermediate position to the locking position.

The guide track 71 has the second rotation portion 77 and the second wing rotating portion 142 which deviates the protrusion from it linear sliding along the longitudinal direction X-X towards the proximal end of the needle to the second stopping area 73. Specifically, second rotation portion 76 and the second wing rotating portion 146 route the protrusion 61, rotating the back-end shield 30 from the linear guiding portion 77 to the second stopping area 73. Finally, the guide track 71 has the stopping area 73 wherein the protrusion 61 is locked after use.

According to one embodiment, the hub 10 comprises two symmetric guides tracks 71 circumferentially spaced apart on the lateral wall 11, one for each protrusion 61. Thanks to the symmetry the sliding and the rotating of the back-end shield is improved avoiding locks during the sliding.

1.-15. (canceled)

16. Safety needle device comprising:
a hub configured to be connected to a pen-injector, the hub comprising a lateral wall and an upper wall which define therebetween a cavity extending between the upper wall and a lower aperture able to receive the pen injector;
a needle attached to the hub, the needle extending along a longitudinal direction between a distal end, configured for insertion into a patient, and an opposed proximal end configured to be connected to the pen injector,
a back-end shield slidable along the longitudinal direction within the cavity of the hub and rotatable about the longitudinal direction, the back-end shield being movable irreversibly from an initial position, before connection of the pen injector to the hub, to a locking position, after removal of the pen injector to the hub passing through an intermediate position,
an elastic member arranged within the cavity and acting between the upper wall and the back-end shield to urge the back-end shield towards the proximal end of the needle along the longitudinal direction,
a locking arrangement comprising at least a first locking member formed on the back-end shield and at least a second locking member, the second locking member cooperating with the first locking member to guide the back-end shield from the initial position to the locking position and to lock the back-end shield in the locking position,
wherein
the second locking member comprises a first guide element formed on the lateral wall of the hub and a second guide element attached to the lateral wall inside the cavity at an intermediate aperture defined between the upper wall and the lower aperture, the first guide element and the second guide element defining a guide track inside the cavity;
the first locking member comprises a protrusion configured to engage the first guide element and the second guide element to slide along the guide track.

17. Safety needle device according to claim 16, wherein the second guide element comprises:
an annular portion configured to engage the lateral wall at the intermediate aperture and defining a passing through channel to allow the sliding of the back-end shield along the longitudinal direction;
a guiding wing projecting from the annular portion towards the distal end inside the cavity and configured to engage the protrusion and guide it from initial position to the locking position.

18. Safety needle device according to claim 17, wherein the guiding wing comprises:
a first wing rotating portion configured to cooperate with the first guiding element to guide and to rotate the back-end shield upon sliding of the back-end shield along the longitudinal axis from the initial position towards the intermediate position;
a second wing rotating portion configured to cooperate with the first guiding element to guide and rotate the back-end shield upon sliding of the back-end shield along the longitudinal axis from the intermediate position towards the locking position.

19. Safety needle device according to claim 17, wherein the annular portion has retaining members configured to engage with the lateral wall at the intermediate aperture.

20. Safety needle device according to claim 16, wherein the first guiding element and the second guiding element define:
a first stopping area configured to engage the protrusion in the initial position of the back-end shield to prevent longitudinal sliding of the back-end shield towards the proximal end of the needle;
a second stopping area configured to engage the protrusion in the locking position of the back-end shield to prevent longitudinal sliding of the back-end shield both towards the distal end and the proximal end of the needle.

21. Safety needle device according to claim 20, wherein:
the first stopping area and the second stopping area are arranged proximal to the intermediate aperture;
the back-end shield in the initial position covers the proximal end of the needle;
the back-end shield in the intermediate position leaves uncovered a first portion of the needle arranged inside the cavity and the proximal end of the needle;
the back-end shield in the locking position covers the proximal end of the needle.

22. Safety needle device according to claim 20, wherein:
the first stopping area is arranged towards the distal end of the needle and the second stopping area is arranged towards the proximal end of the needle;
the back-end shield in the initial position leaves uncovered a second portion of needle arranged inside the cavity and the proximal end of the needle;
the back-end shield in the intermediate position leaves uncovered a first portion of the needle arranged inside the cavity and the proximal end of the needle, the second portion being shorter than the first portion
the back-end shield in the locking position covers the proximal end of the needle.

23. Safety needle device according to claim 19, wherein first guiding element defines:
a third stopping area configured to engage the protrusion in the intermediate position to prevent longitudinal sliding of the back-end shield towards the distal end of the needle;
the third stopping area being spaced apart from the first stopping area and the second stopping area along the longitudinal direction towards the distal end of the needle.

24. Safety needle device according to claim 16, wherein the first guiding element comprises:
a first rotation portion configured to engage the protrusion and cooperating with the second guiding element to rotate the back-end shield upon sliding of back-end shield along the longitudinal axis from the initial position towards the intermediate position;
a second rotation portion configured to engage the protrusion and cooperating with the second guiding element to rotate the back-end shield upon sliding of the back-end shield along the longitudinal axis from the intermediate position towards the locking position.

25. Safety needle device according to claim 16, wherein the first guiding element comprises:
a linear guiding portion configured to engage the protrusion and guide the back-end shield along longitudinal axis preventing rotation of the back-end shield from the initial position towards the intermediate position and from the intermediate position towards the locking position.

26. Safety needle device according to claim 16, wherein:
the hub has an inner surface facing to the cavity and opposed outer surface;
the first guide element comprises a groove formed on the inner surface;
the protrusions are pins.
the elastic member comprises a spring element extending between a distal portion and a proximal portion along the longitudinal axis.

27. Safety needle device according to claim 16, wherein the back-end shield comprises an hollow body extending between a distal portion facing to the upper wall of the hub and a proximal portion facing to the lower aperture of the hub, the hollow body comprising:
a lower wall formed proximal to the distal portion and having an opening configured to allow a portion of the needle and the proximal end of the needle to pass upon moving of the back-end shield between the initial position and locking position;
a lateral wall extending from the lower wall along the longitudinal axis and defining a passing through channel configured to house at least partially the needle;
a retaining wall extending along the longitudinal direction from the lower wall towards the distal end of the needle and surrounding a portion of the lateral wall.

28. Safety needle device according to claim 27, wherein the retaining wall has an inner surface facing the surrounded portion of the lateral wall and an outer surface facing to the cavity of the hub and the protrusions are formed on the outer surface of the back-end shield.

29. Safety needle device according to claim 27, wherein:
the elastic member is interposed between the upper wall of the hub and the lower wall of the back-end shield;
the elastic member surrounds at least partially the needle passing inside the cavity of the hub.

30. Safety needle device according to claim 16, wherein
the hub comprises two symmetric guides tracks circumferentially spaced apart on the lateral wall;
the back-end shield comprises two symmetric protrusions circumferentially spaced apart on the back-end shield,
the second guiding element comprises two symmetric guiding wings circumferentially spaced apart on the annular portion.
